(19) United States
(12) Patent Application Publication
(10) Pub. No.: US 2002/0197218 A1
(43) Pub. Date: Dec. 26, 2002

(54) Photo-crosslinkable nail varnishes free of unsaturated monomers

(75) Inventors: Pascale Bernard, Sucy-en-Brie (FR); Jean Mondet, Aulnay-sous-Bois (FR)

Correspondence Address: STEPTOE & JOHNSON LLP, 1330 Connecticut Ave., N.W., Washington, DC, 20036, US

(73) Assignee: L'OREAL, Paris (FR)

(21) Appl. No.: 10/115,426

(22) Filed: Apr. 4, 2002

(30) Foreign Application Priority Data
Apr. 6, 2001 (FR) 01 04679

(51) Int. Cl. A61K 7/04; C08F 2/46

(52) U.S. Cl. 424/61; 522/182

(57) ABSTRACT

The invention relates to a photo-crosslinkable nail varnish composition that is free of reactive monomers comprising one or more ethylenic double bonds and containing, in a physiologically acceptable medium,

a) one or more polymers comprising ethylenic double bonds, the average number of ethylenic double bonds per polymer molecule being greater than 1, and

b) from 0.1% to 10% by weight, relative to the total weight of polymer (a) comprising ethylenic double bonds, of at least one free-radical photo-initiator,

and also to a cosmetic process for coating nails or false nails using such a composition.

The present invention relates to photo-crosslinkable cosmetic compositions, in particular nail varnishes, free of monomers, and also to a process for depositing a coating crosslinked in situ using these compositions.

Nail varnish compositions may be used as varnish bases, as nail makeup products, as finishing compositions, also known as topcoats, to be applied over the nail makeup product, or alternatively as cosmetic nailcare products. These compositions may be applied either to human nails or to false nails.

Liquid cosmetic compositions which, after depositing on the nail and under the action of an actinic radiation, undergo polymerization and/or crosslinking reactions in situ resulting in polymer networks that are usually crosslinked are known in the field of nail varnishes. Such photo-polymerizable and/or photo-crosslinkable compositions are described, for example, in patents CA 1 306 954 and U.S. Pat. No. 5,456,905.

These compositions generally contain one or more polymers and/or prepolymers comprising polymerizable ethylenic double bonds, one or more reactive monomers containing (a) double bond(s), at least one photo-initiator, optionally one or more agents for inhibiting the premature polymerization of the components, or photo-sensitizers for modifying the absorption spectrum of the photo-initiating system.

The main drawback of these compositions lies in the toxicity of the unsaturated monomers used. Specifically, these highly reactive, low molecular weight molecules readily diffuse into the subjacent and adjacent substrates where they react with the biological molecules.

The Applicant has sought to solve this problem of toxicity of the photo-crosslinkable cosmetic compositions of the prior art by developing photo-crosslinkable cosmetic compositions containing, as sole polymerizable or crosslinkable reactive components, macromolecular compounds, that is to say the reactive components having a molecular mass that is sufficient to prevent them from diffusing towards the neighbouring biological substrates.

Consequently, one subject of the present invention is photo-crosslinkable nail varnish compositions that are free of reactive monomers containing (an) ethylenic double bond(s), containing, in a physiologically acceptable medium,

one or more polymers comprising ethylenic double bonds, the average number of ethylenic double bonds per polymer molecule being greater than 1, and

from 0.1% to 10% by weight, relative to the total weight of polymer comprising ethylenic double bonds, of at least one free-radical photo-initiator.

A subject of the invention is also a cosmetic process for coating nails or false nails using these compositions.

As mentioned above, the average number of ethylenic double bonds borne by the polymers forming the crosslinked polymer network of the final hardened coating must be greater than 1. The reason for this is that a polymerizable system consisting of molecules (monomers, oligomers or polymers) each bearing a single double bond forms, after reaction of all of the double bonds, a macromolecular system containing linear or branched but not crosslinked chains. Only the presence of a certain fraction of molecules bearing at least two double bonds and consequently acting as crosslinking agents makes it possible to obtain a crosslinked polymer system.

In the practice of the present invention, the average number of functions per polymer molecule is preferably greater than 2 and better still greater than 3. As explained above, the harmlessness of the photo-crosslinkable cosmetic compositions of the present invention is due to the fact that the polymers comprising one or more ethylenic double bonds have a molecular mass that is sufficient to prevent them from diffusing towards and into the neighbouring biological substrates. The expression “sufficient molecular mass” means a weight-average molecular mass of greater than or equal to 500. In particular, the weight-average molecular mass may range from 500 to 10,000.

The harmlessness of the compositions will be proportionately greater the higher the mass of the photo-crosslinkable polymers.

In one preferred embodiment of the invention, the photo-crosslinkable polymers have a weight-average molecular mass of greater than or equal to 1000, especially ranging from 1000 to 10,000 and preferably ranging from 2000 to 10,000.

The polymers bearing ethylenic double bonds and having a suitable molar mass for the present invention are known in the art and commercially available.

Examples that may be mentioned of photo-crosslinkable polymers that may be used in the cosmetic compositions of the present invention include:

a) ethylenically unsaturated polyesters:

This is a group of polymers of polyester type containing one or more ethylenic double bonds, randomly distributed in the main polymer chain. These unsaturated polyesters are obtained by polycondensation of a mixture

of linear or branched aliphatic or cycloaliphatic dicarboxylic acids especially containing from 3 to 50 carbon atoms and preferably from 3 to 20 carbon atoms, such as adipic acid or sebacic acid, of aromatic dicarboxylic acids especially containing from 8 to 50 carbon atoms and preferably from 8 to 20 carbon atoms, such as phthalic acids, especially terephthalic acid, and/or of dicarboxylic acids derived from ethylenically unsaturated fatty acid dimers such as the oleic or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,

of linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexanedimethanol, of aromatic diols containing from 6 to 50 carbon atoms and preferably from 6 to 20 carbon atoms, such as bisphenol A and bisphenol B, and/or of diol dimers obtained from the reduction of fatty acid dimers as defined above, and

of one or more dicarboxylic acids or anhydrides thereof comprising at least one polymerizable ethylenic double bond and containing from 3 to 50 carbon atoms and preferably from 3 to 20 carbon atoms, such as maleic acid, fumaric acid or itaconic acid.

b) polyesters containing (meth)acrylate side groups and/or end groups:

This is a group of polymers of polyester type obtained by polycondensation of a mixture

of linear or branched aliphatic or cycloaliphatic dicarboxylic acids especially containing from 3 to 50 carbon atoms and preferably from 3 to 20 carbon atoms, such as adipic acid or sebacic acid, of aromatic dicarboxylic acids especially containing from 8 to 50 carbon atoms and preferably from 8 to 20 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,

of linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexanedimethanol, of aromatic diols containing from 6 to 50 carbon atoms and preferably from 6 to 20 carbon atoms, such as bisphenol A and bisphenol B, and

of at least one ester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glycerol methacrylate.

These polyesters differ from those described above in point a) by the fact that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50,000: molar mass 1500, on average 6 acrylate functions per molecule)

c) polyurethanes and/or polyureas containing (meth)acrylate groups, obtained by polycondensation

of aliphatic, cycloaliphatic and/or aromatic diisocyanates, triisocyanates and/or polyisocyanates especially containing from 4 to 50 and preferably from 4 to 30 carbon atoms, such as hexamethylene diisocyanate, isophorone diisocyanate, toluene diisocyanate, diphenylmethane diisocyanate or isocyanurates of formula

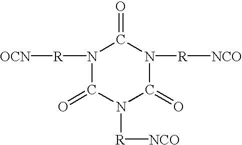

resulting from the trimerization of 3 molecules of diisocyanates OCN—R—CNO, in which R is a linear, branched or cyclic hydrocarbon-based radical comprising from 2 to 30 carbon atoms;

of polyols, especially of diols, free of polymerizable ethylenic unsaturations, such as 1,4-butanediol, ethylene glycol or trimethylolpropane, and/or of aliphatic, cycloaliphatic and/or aromatic polyamines, especially diamines, especially containing from 3 to 50 carbon atoms, such as ethylenediamine or hexamethylenediamine, and

of at least one ester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glycerol methacrylate.

Such polyurethane/polyureas containing acrylate groups are sold, for example, under the name SR 368 (tris(2-hydroxyethyl) isocyanurate-triacrylate) or Craynor® 435 by the company Cray Valley, or under the name Ebecryl® by the company UCB (Ebecryl® 210: molecular mass 1500, 2 acrylate functions per molecule, Ebecryl® 230: molecular mass 5000, 2 acrylate functions per molecule, Ebecryl® 270: molecular mass 1500, 2 acrylate functions per molecule, Ebecryl® 8402: molecular mass 1000, 2 acrylate functions per molecule, Ebecryl® 8804: molecular mass 1300, 2 acrylate functions per molecule, Ebecryl® 220: molecular mass 1000, 6 acrylate functions per molecule, Ebecryl® 2220: molecular mass 1200, 6 acrylate functions per molecule, Ebecryl® 1290: molecular mass 1000, 6 acrylate functions per molecule, Ebecryl® 800: molecular mass 800, 6 acrylate functions per molecule). Mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB.

d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10,000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley.

e) epoxyacrylates obtained by reaction between

at least one diepoxide chosen, for example, from:

bisphenol A diglycidyl ether,

a diepoxy resin resulting from the reaction between bisphenol A diglycidyl ether and epichlorohydrin,

an epoxy ester resin containing α,ω-diepoxy end groups resulting from the condensation of a dicarboxylic acid containing from 3 to 50 carbon atoms with a stoichiometric excess of (i) and/or (ii), and

an epoxy ether resin containing α,ω-diepoxy end groups resulting from the condensation of a diol containing from 3 to 50 carbon atoms with a stoichiometric excess of (i) and/or (ii),

natural or synthetic oils bearing at least 2 epoxide groups, such as epoxidized soybean oil, epoxidized linseed oil or epoxidized vernonia oil,

a phenol-formaldehyde polycondensate (Novolac® resin), the end groups and/or side groups of which have been epoxidized,

and

one or more carboxylic acids or polycarboxylic acids comprising at least one ethylenic double bond in the α,β-position relative to the carboxylic group, for instance (meth)acrylic acid or crotonic acid or esters of (meth)acrylic acid and of a diol or polyol containing from 2 to 20 carbon atoms and preferably from 2 to 6 carbon atoms, such as 2-hydroxyethyl (meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel.

f) poly(C₁₋₅₀ alkyl (meth)acrylates) comprising at least two functions containing an ethylenic double bond borne by the hydrocarbon-based side chains and/or end chains.

Such copolymers are sold, for example, under the names IRR® 375, OTA® 480 and Ebecryl® 2047 by the company UCB.

g) polyorganosiloxanes containing (meth)acrylate or (meth)acrylamide groups obtained, respectively,

by esterification, for example with (meth)acrylic acid, of polyorganosiloxanes, preferably of polydimethylsiloxanes (PDMSs), bearing hydroxyl end groups and/or side groups,

by amidation, for example with (meth)acrylic acid, of polyorganosiloxanes bearing primary or secondary amine side groups and/or end groups.

Hydroxylate PDMSs that may be mentioned in particular are PDMSs comprising at least two C₁₋₆ hydroxyalkyl groups and dimethicone copolyols with hydroxyl side groups or end groups.

Esterifiable α,ω-dihydroxylated polydimethylsiloxanes are sold under the names Tegomer® H-Si 2111 and Tegomer® H-Si 2311 by the company Goldschmidt. α,ω-Diacrylate polydimethylsiloxanes are available from the company Shin-Etsu under the references X-22-164 B and X-22-164 C.

Amino PDMSs that may be mentioned in particular are PDMSs comprising at least 2 C₁₋₁₀ aminoalkyl groups, for example, the aminosilicone sold under the name Q2-8220 by the company Dow Corning.

Advantageously, the silicone polymers of this group are used as a mixture with one or more polymers of other groups a) to f) described above, especially to modify the hydrophobic nature of the final composition.

h) perfluoropolyethers containing acrylate groups obtained by esterification, for example with (meth)acrylic acid, of perfluoropolyethers bearing hydroxyl side groups and/or end groups. Such α,β-diol perfluoropolyethers are described especially in EP-A-1 057 849 and are sold by the company Ausimont under the name Fomblin® Z Diol.

i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

Dendrimers (from the Greek dendron=tree) are “arborescent”, that is to say highly branched, polymer molecules invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech. Hyperbranched polymers are polycondensates, generally of polyester, polyamide or polyethyleneamine type, obtained from multifunctional monomers, which have an arborescent structure similar to that of dendrimers but are much less regular than dendrimers (see, for example, WO-A-93/17060 and WO 96/12754). The company Perstorp sells hyperbranched polyesters under the name Boltorn®. Hyperbranched polyethylene-amines will be found under the name Comburst® from the company Dendritech. Hyperbranched poly(esteramides) containing hydroxyl end groups are sold by the company DSM under the name Hybrane®.

These hyperbranched dendrimers and polymers esterified or amidated with acrylic acid and/or methacrylic acid are distinguished from the polymers described in points a) to h) above by the very large number of ethylenic double bonds present. This high functionality, usually greater than 5, makes them particularly useful by allowing them to act as “crosslinking nodes”, that is to say sites of multiple crosslinking. In one preferred embodiment of the invention, these dendritic and hyperbranched polymers will consequently be used in combination with one or more of the polymers and/or oligomers a) to h) above.

As other polymers that may be used according to the invention, it is also possible to use a polyene combined with a polythiol.

The polyene is a polymer such as those defined above in a) to i), or alternatively a polymer comprising at least two allylic end groups and/or side groups. The latter polymer can result from the polycondensation

of linear or branched aliphatic or cycloaliphatic diols especially containing from 2 to 50 carbon atoms and preferably from 2 to 20 carbon atoms, such as ethylene glycol, diethylene glycol, propylene glycol, 1,4-butanediol or cyclohexanedimethanol, of aromatic diols containing from 6 to 50 carbon atoms and preferably from 6 to 20 carbon atoms, such as bisphenol A or bisphenol B,

of linear or branched aliphatic or cycloaliphatic dicarboxylic acids especially containing from 3 to 50 carbon atoms and preferably from 3 to 20 carbon atoms, such as adipic acid or sebacic acid, of aromatic dicarboxylic acids especially containing from 8 to 50 carbon atoms and preferably from 8 to 20 carbon atoms, such as phthalic acids, especially terephthalic acid, and/or of dicarboxylic acids obtained from ethylenically unsaturated fatty acid dimers, or of aliphatic, cycloaliphatic and/or aromatic diisocyanates, triisocyanates and/or polyisocyanates especially containing from 4 to 50 and preferably from 4 to 30 carbon atoms,

in the presence of a monoalcohol bearing 2 groups containing allylic unsaturation, for instance trimethylolpropane diallyl ether. This monoalcohol acts as a chain terminator.

The polythiol is an oligomeric or polymeric organic compound bearing more than 2 thiol side groups or end groups. For example, polythiols result from the esterification of a polyol, especially containing from 4 to 10 carbon atoms, and of a monocarboxylic acid containing a thiol function especially containing from 2 to 6 carbon atoms, such as thioglycolic acid or 2-mercaptopropionic acid. Polythiols that may be mentioned include trimethylolpropane tris(β-mercapto-propionate) and pentaerythrityl tetrathioglycolate.

The polymers comprising ethylenic double bonds described above generally represent from 0.1% to 99% by weight and preferably from 1% to 90% by weight of the photo-crosslinkable cosmetic composition of the present invention.

The photo-initiators that may be used in the cosmetic compositions of the present invention are also known in the art and are described, for example, in the following articles, the content of which forms an integral part of the present patent application: “Les photoinitiateurs dans la reticulation des revêtements [Photo-initiators in the crosslinking of coatings]”, G. Li Bassi, Double Liaison—Chimie des Peintures, No. 361, November 1985, pages 34-41; “Applications industrielles de la polymérisation photoinduite [Industrial applications of photo-induced polymerization]”, Henri Strub, L'Actualité Chimique, February 2000, pages 5-13; and “Photopolymères: considérations théoriques et réaction de prise [Photopolymers: theoretical considerations and setting reaction]”, Marc, J. M. Abadie, Double Liaison—Chimie des Peintures, No. 435-436, 1992, pages 28-34.

These photo-initiators encompass

the α-hydroxy ketones sold, for example, under the names Irgacure® 184, 1173, 2959, 149, 1000, 5000 and 4265 by the company Ciba,

the α-amino ketones sold, for example, under the names Irgacure® 907 and 369 by the company Ciba,

the chloroacetophenones sold, for example, under the names Trigonal® P by the company Akzo and Sandoray® 1000 by the company Sandoz,

the aromatic ketones sold, for example, under the names Daitocure® by Dainippon, Uvecryl® P 36 by UCB, Esacure® TZT by Lamberti and Quantacure® ITX by Ward Blenkinsop. Mention may also be made of thioxanthones (for example Ultracure® DXT from Sherwin Williams) and quinones (2-ethylanthraquinone from BASF). These aromatic ketones usually require the presence of a hydrogen-donating compound such as tertiary amines and more particularly alkanolamines,

the benzoin ethers sold, for example, under the name Esacure® EB-3 by the company Lamberti and under the name Trigonal® 14 by Akzo,

the α-dicarbonyl derivatives, the most common representative of which is benzyl dimethyl ketal, sold under the name Irgacure® 651 by Ciba. Other commercial products are sold by the company Lamberti under the name Esacure® KBO and by the company Ward Blenkinsop under the name Quantacure² PDO,

the acylphosphine oxides such as, for example, the bis-acylphosphine oxides (BAPOs) sold, for example, under the names Irgacure® 819, 1700, 1800 and 1850 and Darocur® 4265 by the company Ciba.

One particular group of photo-initiators that may be used in the photo-crosslinkable cosmetic compositions of the present invention is that of copolymerizable photo-initiators. These are molecules comprising both a photo-initiating group capable of a photo-induced free-radical splitting and at least one ethylenic double bond. The photo-initiators of this group have the advantage over the standard photo-initiators mentioned above of being able to be integrated, via the double bond, into the macromolecular system. This possibility reduces the concentration of free residual photo-initiators that have not undergone a photo-induced free-radical cleavage and consequently improves the harmlessness of the cosmetic compositions.

Examples of such copolymerizable photo-initiators that may be mentioned include the benzophenone acrylated derivatives sold by the company UCB under the names Ebecryl® P36 and Ebecryl® P37.

In one preferred embodiment of the invention, polymeric photo-initiators or photo-initiators attached to a molecule of high molecular mass are used. The choice of such a photo-initiator of high mass has the same advantage as the selection of exclusively polymeric copolymerizable components, namely better harmlessness of the photo-crosslinkable cosmetic compositions due to the absence of highly reactive small molecules capable of diffusing into the neighbouring biological substrates. As for the copolymerizable components, the weight-average molecular mass of the photo-initiator is preferably at least equal to 500.

Examples that may be mentioned include the α-hydroxy ketone oligomer corresponding to the following formula:

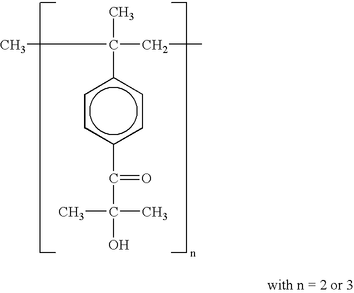

which is sold under the names Esacure® KIP 150 and Esacure® KIP EM by the company Lamberti.

The polymer onto which the photo-initiating group is attached may optionally comprise one or more ethylenic double bonds optionally allowing the integration, into the macromolecular network, of the photo-initiator molecules that have not undergone a photo-induced splitting.

Examples of such photo-initiators of high molecular mass bearing ethylenic double bonds that may be mentioned are those corresponding to the following formulae:

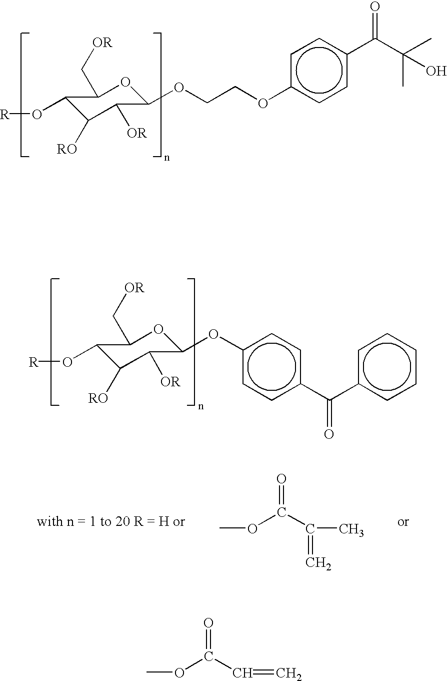

which structures are described in the following articles: S. Knaus, Pure Appl. Chem., A33(7), 869 (1996); S. Knaus, J. Polym. Sci, Part A=Polym. Chem., 33, 929 (1995); and R. Liska, Rad'Tech Europe 97, Lyons, F, 1997, Conference Proceedings.

A mixture of photo-initiators that absorb light at different wavelengths is preferably used in the photo-crosslinkable cosmetic compositions of the present invention. It is thus possible to adapt the absorption spectrum of the photo-crosslinkable cosmetic compositions to the emission spectrum of the light sources used.

The concentration of the photo-initiator(s) used depends on a large number of factors such as, for example, the reactivity of the various components of the mixture, the presence of pigments or colorants, the desired crosslinking density, the intensity of the light source or the exposure time.

In order to obtain satisfactory mechanical properties, the total amount of photo-initiator(s) must be between 0.1% and 10% by weight relative to the total weight of polymer comprising ethylenic double bonds, and an amount of between 0.2% and 5% by weight, relative to the total weight of polymer (a) comprising ethylenic double bonds, will preferably be used.

The photo-crosslinkable cosmetic compositions of the present invention may contain one or more solvents chosen from water and physiologically acceptable organic solvents, among which mention may be made of

a) ketones that are liquid at room temperature, such as methyl ethyl ketone, methyl isobutyl ketone, diisobutyl ketone, isophorone, cyclohexanone and acetone,

b) alcohols that are liquid at room temperature, such as ethanol, isopropanol, diacetone alcohol, 2-butoxyethanol or cyclohexanol,

c) glycols that are liquid at room temperature, such as ethylene glycol, propylene glycol, pentylene glycol and glycerol,

d) propylene glycol ethers that are liquid at room temperature, such as propylene glycol monomethyl ether, propylene glycol monomethyl ether acetate and dipropylene glycol mono-n-butyl ether,

e) short-chain esters (containing in total from 3 to 8 carbon atoms), such as ethyl acetate, methyl acetate, propyl acetate, n-butyl acetate and isopentyl acetate,

f) alkanes that are liquid at room temperature, such as decane, heptane, dodecane and cyclohexane,

g) aromatic hydrocarbons that are liquid at room temperature, such as toluene and xylene,

h) silicones that are liquid at room temperature, and

i) mixtures thereof.

The solvent content in the composition may range from 0.1% to 80% by weight relative to the total weight of the composition and preferably from 1% to 60% by weight.

According to one particular embodiment of the composition according to the invention, the composition is free of solvent.

The photo-crosslinkable cosmetic compositions of the present invention may also contain adjuvants and additives commonly used in nail varnishes, chosen especially from pigments and colorants, plasticizers, coalescers, preserving agents, waxes, thickeners, fragrances, UV screening agents, cosmetic nailcare active agents, spreading agents, antifoams and dispersants.

Needless to say a person skilled in the art will take care to select these optional adjuvants and additives such that the advantageous properties of the compositions according to the invention are not, or are virtually not, adversely affected by the envisaged addition.

When the composition comprises pigments and/or colorants, it is convenient in particular to adapt the absorption spectrum of the pigments and/or colorants used to that of the photo-initiators, or, conversely, the absorption spectrum of the photo-initiators to that of the pigments and/or colorants used, so as to avoid these two types of compound absorbing light at the same wavelengths. Specifically, the absorption of light by the pigments and/or colorants would make the photo-initiators present almost totally ineffective beyond a certain depth in the coating.

A subject of the present invention is also a cosmetic process for coating natural nails or false nails.

This process comprises

the application of a photo-crosslinkable cosmetic composition containing, in a physiologically acceptable medium, (a) one or more oligomers and/or polymers comprising ethylenic double bonds, the average number of ethylenic double bonds per oligomer and/or polymer molecule being greater than 1, and (b) from 0.1% to 10% by weight, relative to the total weight of the polymer comprising ethylenic double bonds, of at least one free-radical photo-initiator, to nails or false nails, and

the exposure of the applied film to a radiation of suitable wavelength for a time that is sufficient to induce the crosslinking of the said film.

When the composition comprises a volatile organic solvent or water, the film is exposed to the radiation after evaporating off the solvent or the water.

In one particular embodiment of the process of the present invention, the keratin substrate (nails, false nails, eyelashes, false eyelashes, hair and hair prostheses) onto which is applied the photo-crosslinkable cosmetic composition is already coated with a first non-crosslinkable cosmetic coating, such as a common basecoat or nail varnish coat.

The photo-crosslinkable cosmetic composition thus constitutes a protective coat (topcoat) for the first non-crosslinkable cosmetic coating. The composition may also be applied alone onto the nails to protect them. In this embodiment, the photo-crosslinkable cosmetic composition is preferably uncoloured, that is to say free of pigments and colorants.

The suitable radiations for the crosslinking of the cosmetic compositions of the present invention have a wavelength of between 210 and 600 nm and preferably between 250 and 400 nm. The use of lasers may also be envisaged.

In one preferred embodiment of the invention, a UV lamp is used, and in particular a mercury-vapour lamp, possibly doped with other elements, such as gallium, allowing the emission spectrum of the light source to be modified.

The exposure time of the applied film to the radiation depends on various factors such as the chemical nature and the concentration of the reactive components or the desired crosslinking density.

For nail varnishes, it will generally be sought to obtain satisfactory results for an exposure time of between 1 and 20 minutes and preferably between 2 and 10 minutes.

This hardening time preferably does not correspond to the total crosslinking of the system, that is to say the reaction of all the double bonds present. To ensure good staying power of the nail varnishes over time, it is advantageous to obtain both a high crosslinking density at the surface of the film, ensuring good mechanical strength and low crosslinking at the coating/substrate interface, which is essential for good adhesion of the applied film to the substrate.

### EXAMPLE 1

A photo-crosslinkable nail varnish having the composition below was prepared:

| | |
|---|---|
| Polyester containing acrylate groups sold | 96.5 g |
| under the name Ebecryl ® 83 by the company UCB | |
| Photo-initiator | 3.5 g |
| (Irgacure ® 500, Ciba) | |

The composition is applied to the nails in the form of a film about 50 μm thick and this film is irradiated for 5 minutes with a UV radiation emitted by a “Philips Original UVA” lamp sold by the company Philips, the effective wavelength range of which is from 340 to 365 mm. The lamp is placed about 50 cm away from the nails and, throughout the exposure time, the hands are alternately inclined in one direction and in the other so as to distribute the radiation uniformly over the entire surface of the nails.

A film that is touch-dry, non-sticky, glossy and hard is thus obtained.

### EXAMPLE 2

The process is performed in the manner described in Example 1, using the following cosmetic composition:

| | |
|---|---|
| Polyester containing acrylate groups sold | 96.5 g |
| under the name Ebecryl ® 140 by the company UCB | |
| Photo-initiator | 3.5 g |
| (Irgacure ® 500, Ciba) | |
| Pigments | qs |

A hard, glossy and non-sticky film is thus obtained.

### Example 3

The process is performed in the manner described in Example 1, using the following cosmetic composition:

| | |
|---|---|
| Polyurethane-triacrylate sold under the | 95 g |
| name Craynor ® 435 by the company Cray Valley | |
| Photo-initiator | 3.5 g |
| (Irgacure ® 500, Ciba) | |
| Pigments | qs |

A hard, glossy and non-sticky film is thus obtained.

1. Photo-crosslinkable nail varnish composition containing, in a physiologically acceptable medium,
a) one or more polymers comprising ethylenic double bonds, the average number of ethylenic double bonds per polymer molecule being greater than 1, and
b) from 0.1% to 10% by weight, relative to the total weight of polymer (a) comprising ethylenic double bonds, of at least one free-radical photo-initiator,
characterized in that it is free of reactive monomers with a molecular mass of less than 500 comprising one or more ethylenic double bonds.

2. Photo-crosslinkable nail varnish composition according to claim 1, characterized in that the average number of ethylenic double bonds per polymer molecule is greater than 2 and preferably greater than or equal to 3.

3. Photo-crosslinkable nail varnish composition according to claim 1 or 2, characterized in that the polymer(s) is(are) chosen from:
a) ethylenically unsaturated polyesters obtained by polycondensation
of linear or branched aliphatic or cycloaliphatic dicarboxylic acids especially containing from 3 to 50 carbon atoms and preferably from 3 to 20 carbon atoms, of aromatic dicarboxylic acids especially containing from 8 to 50 carbon atoms and preferably from 8 to 20 carbon atoms, and/or of dicarboxylic acids derived from ethylenically unsaturated fatty acid dimers, all these diacids being free of polymerizable ethylenic double bonds,
of linear or branched aliphatic or cycloaliphatic diols especially containing from 2 to 50 carbon atoms and preferably from 2 to 20 carbon atoms, of aromatic diols containing from 6 to 50 carbon atoms and preferably from 6 to 20 carbon atoms, and/or of diol dimers obtained from the reduction of fatty acid dimers as defined above, and
of one or more dicarboxylic acids or anhydrides thereof comprising at least one polymerizable ethylenic double bond and containing from 3 to 50 carbon atoms and preferably from 3 to 20 carbon atoms,
b) polyesters containing (meth)acrylate side groups and/or end groups obtained by polycondensation
of linear or branched aliphatic or cycloaliphatic dicarboxylic acids especially containing from 3 to 50 carbon atoms and preferably from 3 to 20 carbon atoms, of aromatic dicarboxylic acids especially containing from 8 to 50 carbon atoms and preferably from 8 to 20 carbon atoms, and/or of dicarboxylic acids derived from ethylenically unsaturated fatty acid dimers, all these diacids being free of polymerizable ethylenic double bonds,
of linear or branched aliphatic or cycloaliphatic diols especially containing from 2 to 50 carbon atoms and preferably from 2 to 20 carbon atoms, of aromatic diols containing from 6 to 50 carbon atoms and preferably from 6 to 20 carbon atoms, and
of at least one ester of (meth)acrylic acid and of a diol or polyol containing from 2 to 20 carbon atoms and preferably from 2 to 6 carbon atoms,
c) polyurethanes and/or polyureas containing (meth)acrylate groups obtained by polycondensation
of aliphatic, cycloaliphatic and/or aromatic diisocyanates, triisocyanates and/or polyisocyanates especially containing from 4 to 50 and preferably from 4 to 30 carbon atoms,
of polyols, especially of diols, free of polymerizable ethylenic unsaturations, such as 1,4-butanediol, ethylene glycol or trimethylolpropane, and/or of aliphatic, cycloaliphatic and/or aromatic polyamines, especially diamines, especially containing from 3 to 50 carbon atoms, and
of at least one ester of (meth)acrylic acid and of a diol or polyol containing from 2 to 20 carbon atoms and preferably from 2 to 6 carbon atoms,
d) polyethers containing (meth)acrylate groups obtained by esterification, with (meth)acrylic acid, of the hydroxyl end groups of C₁₋₄ alkylene glycol homopolymers or copolymers,
e) epoxyacrylates obtained by reaction between
at least one diepoxide and
one or more carboxylic acids or polycarboxylic acids comprising at least one ethylenic double bond in the α,β-position relative to the carboxylic group, for instance (meth)acrylic acid, crotonic acid comprising at least one (meth)acrylic double bond, such as (meth)acrylic acid, or esters of (meth)acrylic acid and of a diol or polyol containing from 2 to 20 carbon atoms and preferably from 2 to 6 carbon atoms,
f) poly(C₁₋₅₀ alkyl (meth)acrylates) comprising at least two functions containing an ethylenic double bond borne by the hydrocarbon-based side chains and/or end chains,
g) polyorganosiloxanes containing (meth)acrylate or (meth)acrylamide groups obtained, respectively,
by esterification, for example with (meth)acrylic acid, of polyorganosiloxanes, preferably of polydimethylsiloxanes (PDMSs), bearing hydroxyl end groups and/or side groups,
by amidation, for example with (meth)acrylic acid, of polyorganosiloxanes bearing primary or secondary amine side groups and/or end groups,
h) perfluoropolyethers containing acrylate groups obtained by esterification, for example with (meth)acrylic acid, of perfluoropolyethers bearing hydroxyl side groups and/or end groups,
i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

4. Photo-crosslinkable nail varnish composition according to claim 3, characterized in that it contains one or more of the polymers (a) to (h) in combination with one or more hyperbranched dendrimers and/or polymers containing (meth)acrylate groups (i).

5. Photo-crosslinkable nail varnish composition according to claim 3, characterized in that it contains one or more of the polymers (a) to (h) in combination with a polythiol.

6. Nail varnish composition according to claim 1 or 2, characterized in that the polymer is a polyene comprising at least two allylic end groups and/or side groups, combined with a polythiol, the polyene comprising at least two allylic end groups and/or side groups being obtained by polycondensation
of linear or branched aliphatic or cycloaliphatic diols especially containing from 2 to 50 carbon atoms and preferably from 2 to 20 carbon atoms, of aromatic diols containing from 6 to 50 carbon atoms and preferably from 6 to 20 carbon atoms, and
of linear or branched aliphatic or cycloaliphatic dicarboxylic acids especially containing from 3 to 50 carbon atoms and preferably from 3 to 20 carbon atoms, of aromatic dicarboxylic acids especially containing from 8 to 50 carbon atoms and preferably from 8 to 20 carbon atoms, and/or of dicarboxylic acids obtained from ethylenically unsaturated fatty acid dimers, or of aliphatic, cycloaliphatic and/or aromatic diisocyanates, triisocyanates and/or polyisocyanates especially containing from 4 to 50 and preferably from 4 to 30 carbon atoms,
in the presence of a monoalcohol bearing 2 groups containing allylic unsaturation, for instance trimethylolpropane diallyl ether.

7. Photo-crosslinkable nail varnish composition according to one of the preceding claims, characterized in that the polymer has a weight-average molar mass of greater than 1000 and preferably between 2000 and 10,000.

8. Photo-crosslinkable nail varnish composition according to any one of the preceding claims, characterized in that the concentration of the polymers containing ethylenic double bonds is between 0.1% and 99% by weight and preferably between 1% and 90% by weight relative to the photo-crosslinkable nail varnish composition.

9. Photo-crosslinkable nail varnish composition according to any one of the preceding claims, characterized in that the free-radical photo-initiator is chosen from α-hydroxy ketones, α-amino ketones, chloroacetophenones, aromatic ketones, preferably combined with a hydrogen-donating compound, benzoin ethers, aromatic α-diketones and acylphosphine oxides.

10. Photo-crosslinkable nail varnish composition according to any one of claims 1 to 8, characterized in that the photo-initiator is chosen from copolymerizable photo-initiators comprising at least one photo-initiating group and at least one ethylenic double bond.

11. Photo-crosslinkable nail varnish composition according to any one of claims 1 to 8, characterized in that the photo-initiator is an oligomer or polymer with an average molecular mass at least equal to 500, or a photo-initiator immobilized on a molecule with an average molecular mass at least equal to 500.

12. Photo-crosslinkable nail varnish composition according to claim 11, characterized in that the oligomeric photo-initiator is an oligomer of formula

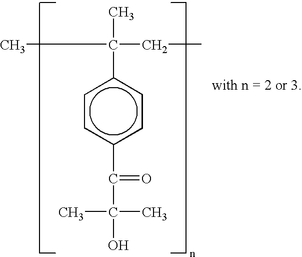

13. Photo-crosslinkable nail varnish composition according to any one of the preceding claims, characterized in that a mixture of several photo-initiators having different absorption wavelengths is used.

14. Photo-crosslinkable nail varnish composition according to any one of the preceding claims, characterized in that the total amount of photo-initiator is between 0.2% and 5% by weight, relative to the total weight of polymer comprising ethylenic double bonds.

15. Photo-crosslinkable nail varnish composition according to any one of the preceding claims, characterized in that it contains one or more solvents chosen from water and physiologically acceptable organic solvents such as ketones, alcohols, glycols, propylene glycol ethers, short-chain esters, alkanes, aromatic hydrocarbons and silicones, that are all liquid at room temperature, and mixtures thereof.

16. Photo-crosslinkable nail varnish composition according to any one of the preceding claims, characterized in that it also contains adjuvants and additives commonly used in nail varnishes, chosen especially from pigments and colorants, plasticizers, coalescers, preserving agents, waxes, thickeners, fragrances, UV screening agents, cosmetic nailcare active agents, spreading agents, antifoams and dispersants.

17. Process for coating nails and false nails, comprising
the application of a photo-crosslinkable nail varnish composition according to any one of claims 1 to 16 to the nails or false nails, and
the exposure of the applied film to a radiation of suitable wavelength for a time that is sufficient to induce the crosslinking of the said film.

18. Process according to claim 17, characterized in that the nails or false nails are already coated with a first cosmetic coating that is not radiation-crosslinkable.

19. Process according to either of claims 17 and 18, characterized in that the radiation has a wavelength of between 210 and 600 nm.

20. Process according to any one of claims 17 to 19, characterized in that the exposure time is between 1 and 20 minutes and preferably between 2 and 10 minutes.
